(19) United States
(12) Patent Application Publication
(10) Pub. No.: US 2010/0075930 A1
(43) Pub. Date: Mar. 25, 2010

(54) Salicylic salt compositions and related method of treatment of cardiac conditions

(76) Inventor: Yoel Ovil, Johannesburg (ZA)

Correspondence Address: Dara L. Onofrio, Esq.,;c/o ONOFRIO LAW, Suite 225, 15 N . Mill Street, Nyack, NY, 10960, US

(21) Appl. No.: 12/584,247

(22) Filed: Sep. 2, 2009

Related U.S. Application Data
(60) Provisional application No. 61/190,875, filed on Sep. 3, 2008.

(51) Int. Cl. A61K 31/60

(52) U.S. Cl. 514/159

(57) ABSTRACT

A method to deliver salicylic acid to the blood comprised of sublingual administration of a salicylic salt and specifically a method to treat cardiac conditions including myocardial infarctions and angina, comprising sublingual administration of a salicylic salt to a patient suffering from a cardiac condition, wherein said salicylic salt is either a liquid spray or tablet; such that a therapeutic dose of salicylic acid is delivered to the blood of the patient in less than 4 minutes.

This application claims the benefit of U.S. provisional application No. 61/190,875 filed Sep. 3, 2009, which is incorporated herein in its entirety by reference.

### FIELD OF THE INVENTION

The present invention relates to salicylic salt compositions which are administered sublingually in a patient to deliver salicylic acid to the blood almost immediately and without adverse gastric side effects. In particular, the invention provides a choline salicylate spray or choline salicylate tablet which is administered sublingually in a patient.

### BACKGROUND OF THE INVENTION

Aspirin (acetyl salicylic acid) in low doses, 75-100 mg daily, has been proven to reduce coronary events by 50%, mainly by its action as a carboxylic oxygenase inhibitor leading to reduced platelets adhesiveness thus preventing clot formation.

The long term chronic use of aspirin however, carries the risk of gastrointestinal bleeding mainly because of it's local effect as an acid which increases the acidity in the stomach as well as systematic effect of the drug i.e. decreased stomach lubrication due to the fact that the drug is anti prostaglandin secretion. Of the above two main mechanisms leading to gastrointestinal bleeding it has been shown that the local effect of aspirin is the main one. Thus, prevention of aspirin touching the stomach will eventually reduce notably, the life threatening complication i.e. gastrointestinal bleeding. Trials to coat the aspirin thus avoiding touch between the stomach and the acid (Ecotrin 81 mg) failed to decrease the rate of the gastrointestinal complication.

The gravity of gastrointestinal complications from aspirin use is severe. More than 22% of patients taking aspirin develop ulcers, perforations, bleeding, obstruction, stricture and/or enteropathy.

High dose aspirin (acetyl salicylic acid) has been demonstrated to be highly effective in acute myocardial infarction in terms of anginal pain disappearance and infarct size limitation. These clinical benefits are attributed to platelet aggregation inhibition (prevention of clot formation) and the ability of aspirin to enhance the anaerobic cell ventilation and to block the aerobic ventilation that has been interrupted due to lack of oxygen in cases of acute myocardial infarction. The main drawback of aspirin treatment in acute myocardial infarction is the time lag needed for the body to transfer the aspirin (acetyl salicylic acid) to salicylic acid which is the active ingredient of aspirin. This metabolic process is called de-acetylation (breaking off the acetyl group from the acetyl salicylic acid (aspirin) and it occurs in the liver. This process is time consuming, taking at least 2½ hours, and delays the action of the drug.

U.S. Pat. No. 3,069,321 to Broh-Kahn discloses a choline salicylate composition and methods of use. The composition is administered in an aqueous media for liquid oral use and is faster acting than aspirin but still has gastric complications.

The invention provides a sublingual method of administration of the active ingredient of aspirin-salicylic acid to blood, allowing the fastest mode of action and eliminates gastrointestinal complications.

The oral administration of choline salicylate will lead the salicylic acid blood level peak to occur in 2 hours after administration. The invention, sublingual method, the salicylic acid blood level peak will occur in 4 minutes after administration. This fast action is unprecedented. In acute myocardial infarcts the level of salicylic acid in the blood is mandatory to instantly stop the process of myocardial necrosis i.e. myocardial infarct. The mechanism that is involved in such process is, as mentioned earlier, enhancing anaerobic ventilation (lipolyses) and blocking the aerobic ventilation that was interrupted because of lack of oxygen. This property of fast acting and high blood level of salicylic acid is essential to limit the infarct size and save millions of lives.

The compositions of the invention and administration thereof abolish gastrointestinal bleeding and allow the active ingredient, salicylic acid, to get to the blood as soon as possible by bypassing the gastrointestinal tract. It is absorbed through either the mouth mucosa or rectal mucosa straight to the blood stream.

The sublingual administration reduces gastrointestinal complications due to the total avoidance between the drug and the gastrointestinal tract. Choline salicylate as a salt syrup (compared to acid tablet) has already reduced notably (by 40%) the gastrointestinal complications due to its salt nature and not acid nature. The present invention provides a further advantage of eliminating gastrointestinal complications and allowing safer chronic use of the drug.

Although it has been described to use the invention compositions and treatment of a patient in acute situations, the invention also provides a safe means to prevent cardiac conditions. It is possible to provide daily administration of the compositions without adverse side effects and to increase the cardiac health of the individual.

An advantage of the present invention is in the delivery of salicylic acid to the blood stream in the fastest mode (4 minutes), thus preventing further damage to the myocardium, limiting the infarct size. The invention is a life saving composition for all to use.

### SUMMARY OF THE INVENTION

In the present invention, these purposes, as well as others which will be apparent, are achieved by providing a method to deliver salicylic acid to the blood comprised of sublingual administration of a salicylic salt, wherein said salicylic salt is either a liquid spray or tablet.

The liquid spray is 80% salicylic salt and 20% water. This solution is stable and the salicylic salt does not break down and react with the water until administration to a patient. This is due to pH levels, the solution while being stored is not in an acidic environment and thus will not react until sprayed under the mouth of the patient. The environment there is acidic, having a range between 7.38 and 7.45. The salicylic salt breaks down to salicylic acid which is readily absorbed under the tongue of the individual.

The tablet is 100% salicylic salt and is absorbed by the individual when placed under the tongue. The tablet can also include additional materials to enhance the taste of the tablet but in no way would these materials interfere with the absorption of the salicylic acid.

Although it is preferred to administer the invention compositions by mouth, it is possible to administer them through the rectal mucosa.

In all embodiments a therapeutic amount of salicylic acid is delivered to the blood in 4 minutes.

Although all salicylic salts are covered by the invention, the preferred salicylic salt is choline salicylate.

The invention also provides a method to treat cardiac conditions including myocardial infarctions and angina, comprising sublingual administration of a salicylic salt to a patient suffering from a cardiac condition, wherein said salicylic salt is either a liquid spray or tablet; such that a therapeutic dose of salicylic acid is delivered to the blood of the patient in less than 4 minutes.

Other objects, features and advantages of the present invention will be apparent when the detailed description of the preferred embodiments of the invention are considered with reference to the drawings, which should be construed in an illustrative and not limiting sense.

### BRIEF DESCRIPTION OF THE DRAWINGS

FIG. 1A is an illustration of the choline salicylate reaction with water; and

FIG. 1B illustrates the formula of choline salicylate.

### DETAILED DESCRIPTION OF THE INVENTION

In accordance with the present invention a method to rapidly deliver salicylic acid to the blood is provided. The invention accomplishes this by sublingual administration of a salicylic salt, where the salicylic salt is either a liquid spray or tablet.

Looking at the chemical structure of acetyl salicylic acid it appears that the molecule is not polar and therefore not soluble in water, this is the main reason why it can not be absorbed through the mouth or rectal mucosa.

The invention compositions in all their embodiments i.e. aerosol, tablet, suppository, IV fluids, is made of salicylic salts which are water soluble. In the preferred embodiment choline salicylate is used, but any other water soluble salicylate can be used.

Choline salicylate is preferred based on the following characteristics.
1. Choline salicylate is the most water soluble salicylate known.
2. Choline salicylate has been in clinical use as choline salicylate syrup which is absorbed through the gastro intestinal tract and has been shown to have no major side effects.
3. Choline salicylate in concentration of 80% (20% Water and 80% choline salicylate) is an optimal solution that can be delivered through a metered spray bottle, easily to be sprayed under the tongue to be absorbed through the mouth mucosa.
4. Choline salicylate in concentration of 95% (5% water and 95% Choline Salicylate) is a solid substance highly soluble in water that can be used for the production of sublingual tablets or rectal suppositories.
5. The clinical use of the syrup choline salicylate absorbed in the intestine has not determined major side effects.

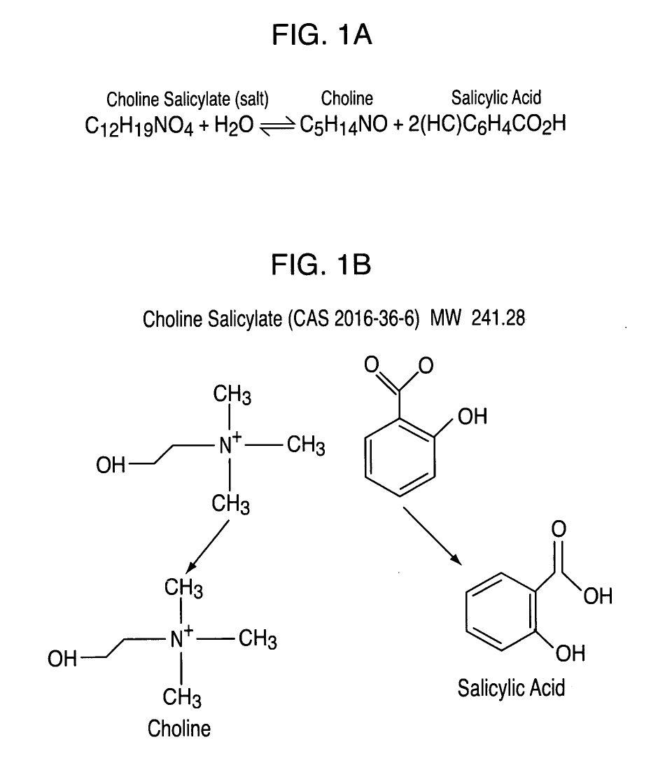

FIG. 1A illustrates the reaction of choline salicylate (salt) with water and FIG. 1B illustrates the chemical formula of choline salicylate having a molecular weight of 241.28 and a melting temperature of approximately 37 degrees Celcius, which is the human body temperature.

The liquid spray is 80% salicylic salt and 20% water; and the tablet is 100% salicylic salt.

The liquid solution is stable and the salicylic does not break down and react with the water until administration to a patient. This is due to pH levels, the solution while being stored is not in an acidic environment and thus will not react until sprayed under the mouth of the patient. The environment there is acidic, having a range between 7.38 and 7.45. The salicylic salt breaks down to salicylic acid which is readily absorbed under the tongue of the individual and quickly finds its way into the blood stream.

The present invention will be illustrated in more detail by the following examples without limiting the scope in any way.

### Example 1

In order to assess the ability and the rate of absorption of choline salicylate liquid under the tongue, the following experiment was conducted:

A preparation of choline salicylate liquid (80% choline salicylate and 20% water) was introduced in a metered spray bottle. It was calculated that each “puff” contained 50 mg of pure choline salicylate.

A 75 kg pig was anaesthetised and 20 “puffs” of proposed spray were administered under the pig's tongue. Special care was taken to keep the head in erect position so there will be no spillage or swallowing of the material.

Blood level of salicylic acid (active ingredient of Aspirin-acetyl salicylic acid) were taken every 3 minutes, then every 15 minutes. Results showed that the level of salicylic acid rose abruptly within 4 minutes after administration.

### Example 2

In order to assess the ability and the rate of absorption of a choline salicylate tablet under the tongue, the following experiment was conducted:

1000 mg of choline salicylate were administered under the tongue. Table 1 below indicates the levels of salicylic acid in the blood measured with a Cobus Integra 400. The level of salicylic acid in the blood after 4 minutes was 21 mg/Lt. Assuming the blood volume is 5 litres we can conclude that 100 mg (therapeutical level) of salicylic acid were presented and available to act 4 minutes after administration.

In comparing this fast availability of salicylic acid in the blood it has been documented that after digestion of 1000 mg of Aspirin (acetyl salicylic acid) the level of salicylic acid in the blood will reach 100 mg/Lt after 2 hours only.

TABLE 1

| Sample | | Conversion | | |
|---|---|---|---|---|
| No. | Time (min.) | factor | mmol/l | mg/l |
| 1 | 0 | 0.00724 | 0.111 | 15.331 |
| 2 | 2 | 0.00724 | 0.124 | 17.127 |
| 3 | 4 | 0.00724 | 0.155 | 21.409 |
| 4 | 6 | 0.00724 | 0.132 | 18.232 |
| 5 | 8 | 0.00724 | 0.096 | 13.260 |
| 6 | 10 | 0.00724 | 0.089 | 12.293 |
| 7 | 12 | 0.00724 | 0.074 | 10.221 |
| 8 | 14 | 0.00724 | 0.067 | 9.254 |
| 9 | 19 | 0.00724 | 0.039 | 5.387 |
| 10 | 24 | 0.00724 | 0.023 | 3.177 |
| 11 | 29 | 0.00724 | 0.040 | 5.525 |
| 12 | 34 | 0.00724 | 0.036 | 4.972 |
| 13 | 39 | 0.00724 | 0.045 | 6.215 |
| 14 | 44 | 0.00724 | 0.054 | 7.459 |

### Example 3

In order to assess the ability and the rate of absorption of salicylic acid to the human blood stream by sublingual administration of a salicylic salt, the following experiment was conducted in the laboratory:

A preparation of choline salicylate liquid (80% choline salicylate and 20% water) was tested under various pH conditions. The standing solution is stable and non-reactive. However when the pH of the solution was modified to 7.38 to 7.45, which corresponds to the pH conditions of the human mouth, the salicylic salt breaks down and reacts with the water to release the salicylic acid which is readily absorbed under the tongue a patient.

A tablet preparation of 100% salicylic salt was also tested under various temperature conditions. The tablet was found to melt at 37 degrees Celcius, which corresponds to the normal human body temperature. Thus, when the salicylic salt tablet is placed under the tongue the salicylic acid is released and absorbed into the blood stream.

From the examples above, it is concluded that the administration method of the invention is superior to the regular gastro intestinal absorption due to:
a. fast rising of blood level
b. the invention method of administration will allow high levels of salicylic acid in the blood, bypassing the gastrointestinal system, thus preventing the fatal complications of gastrointestinal bleeding.

In addition, the rapid delivery of salicylic acid to the human blood stream provides extraordinary benefits in treating myocardial infarctions and angina, as well as use to prevent damaging cardiac conditions.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A method to deliver salicylic acid to the blood comprised of sublingual administration of a water soluble salicylic salt.

2. The method according to claim 1, wherein said salicylic salt is in a vehicle that is selected from the group consisting of liquid, liquid spray, aerosol, tablet, suppository and IV fluids.

3. The method according to claim 1, wherein said water soluble salicylic salt has a pH in the range of 7.3 to 7.5.

4. The method according to claim 2, wherein said liquid spray is 80% salicylic salt and 20% water.

5. The method according to claim 2, wherein said tablet is 100% salicylic salt and melts at 37 degrees Celcius.

6. The method according to claim 1, wherein the salicylic acid is delivered to the blood in 4 minutes.

7. The method according to claim 1, wherein said salicylic salt is choline salicylate.

8. A method to treat cardiac conditions including myocardial infarctions and angina, comprising:
sublingual administration of a water soluble salicylic salt to a patient suffering from a cardiac condition, wherein said salicylic salt is in a vehicle that is selected from the group consisting of liquid, liquid spray, aerosol, tablet, suppository and IV fluids is either a liquid spray or tablet;
such that a therapeutic dose of salicylic acid is delivered to the blood of the patient in less than 4 minutes.

9. The method according to claim 8, wherein said liquid spray is 80% salicylic salt and 20% water.

10. The method according to claim 9, wherein said spray is absorbed quickly under the patients tongue.

11. The method according to claim 8, wherein said tablet is 100% salicylic salt.

12. The method according to claim 11, wherein said tablet dissolves at 37 degrees Celcius and is absorbed quickly under the patients tongue.

13. The method according to claim 8, wherein said salicylic salt is choline salicylate.

14. The method according to claim 8, wherein said water soluble salicylic salt has a pH in the range of 7.3 to 7.5.
